(19) United States
(12) Patent Application Publication
(10) Pub. No.: US 2002/0166134 A1
(43) Pub. Date: Nov. 7, 2002

(54) Cardiomyocytes with enhanced proliferative potenial, and methods for preparing and using same

(76) Inventors: Loren J. Field, Indianapolis, IN (US); Kishore Babu S. Pasumarthi, Indianapolis, IN (US)

Correspondence Address: Kenneth A. Gandy, Woodard, Emhardt, Naughton, Moriarty & McNett, Bank One Center/Tower, Suite 3700, 111 Monument Circle, Indianapolis, IN, 46204-5137, US

(21) Appl. No.: 10/024,066

(22) Filed: Dec. 18, 2001

Related U.S. Application Data
(63) Continuation of application No. PCT/US00/16827, filed on Jun. 19, 2000.
(60) Provisional application No. 60/139,942, filed on Jun. 18, 1999.

(51) Int. Cl. A61K 48/00; A01K 67/00; C12N 15/87

(52) U.S. Cl. 800/8; 424/93.21; 514/44; 435/455

(57) ABSTRACT

Described are methods in vitro and in vivo which involve the use of increased cyclin D2 activity to activate the cell cycle of cardiomyocytes as a baseline measure and/or in response to stimuli. Also described are vectors useful for these purposes, and cardiomyocyte cells exhibiting an activated cell cycle. Transgenic cyclin D2 animal models are also described.

### REFERENCE TO RELATED APPLICATION

This application claims the benefit of U.S. patent application Ser. No. 60/139,942 filed Jun. 18, 1999, which is hereby incorporated herein by reference in its entirety.

### BACKGROUND

The present invention relates generally to cardiomyocytes and their use, and in particular aspects to cardiomyocytes containing introduced nucleic acid which encodes a cyclin D2 protein and having increased proliferative capacity, and to methods of making and using such cardiomyocytes.

It is well established that adult mammalian cardiomyocytes exhibit very limited proliferative potential. Studies have shown, for example, that the labeling index for cardiomyocytes in normal adult hearts is less than 0.006% as measured using tritiated thymidine assays with transgenic mice expressing a cardiomyocyte-restricted β-galactosidase reporter to mark cardiomyocyte nuclei. Soonpaa, M. H., and Field, L. J., Am. J. Physiol. 266:H1439-1445 (1997). As a result, the mammalian myocardium lacks significant capacity for regenerative growth.

Regenerative myocardial growth has enormous therapeutic potential, for example to address many forms of cardiovascular disease characterized by cardiomyocyte death with an ensuing loss of myocardial function. Consequently, efforts have been made to develop strategies to induce cardiomyocyte proliferation. A number of factors have been shown to augment cardiomyocyte DNA synthesis in vitro (Oberpriller, J. O., et al., The Development and Regenerative Potential of Cardiac Muscle, Hardwood Academic Publishers, Chur, Switzerland/New York (1991)). However, no factor examined to date has proven to induce sustained proliferation of differentiated cardiomyocytes in fetal or adult cultures.

The onset of gene transfer techniques has spurred various studies to test the ability of a specific gene product to augment myocardial proliferation in vitro or in vivo. For example, such studies have been carried out involving the forced expression of v-myc (Saule, S. et al., Proc. Natl. Acad. Sci. USA 84:7982-7986 (1987); Engelmann, G. L. et al., J. Mol. Cell. Cardiol. 25:197-213 (1993)), c-myc (Jackson, T. et al., Mol. Cell. Biol. 10:3709-3716 (1990); Jackson, T. et al., Mol. Cell. Biochem. 104:15-19 (1991)), IGF-1B (Reiss, K. et al., Proc. Natl. Acad. Sci. USA 93:8630-8635 (1996)), E1A (Kirshenbaum, L. A., and M. D. Schneider, J. Biol. Chem. 270:7791-7794 (1995)), and SV40 T antigen (Field, L. J., Science 239:1029-1033 (1988); Katz, E., et al., Am. J. Physiol. 262:H1867-H1876 (1992)).

Although these research efforts have demonstrated that forced expression of cellular protooncogenes or transforming oncogenes from DNA tumor viruses can promote cardiomyocyte DNA synthesis, and in some cases proliferation, progress on the identification of genes which might be useful to induce regenerative myocardial growth has been difficult and slow.

The mammalian cell cycle has been an area of considerable research interest for many years. This cycle includes a first phase of growth known as the G1 phase, and proceeds then to the S phase, in which DNA replication occurs. The S phase is followed by a second phase of growth known as the G2 phase where cells increase in mass. The cycle terminates in the M phase, which involves nuclear division and cytokinesis. Passage through this cell cycle is regulated at several checkpoints. A highly orchestrated cascade ensures that all requisite activities (genome reduplication, DNA repair, chromosome segregation, etc.) are completed before the initiation of the next step of the cell cycle. The presence of multiple checkpoints can also provide mechanisms for identifying and eliminating of aberrantly growing or genetically compromised cells.

Transition through the cell cycle checkpoints is regulated in part by the activity of a family of protein kinases, the cyclin dependent kinases (CDKs), and their activating partners, the cyclins. In most instances, the initiation of DNA synthesis requires transit through the so-called restriction point, which is at the G1→S boundary of the cell cycle. Transit through this restriction point is to a large extent regulated by CDK4 and the D-type cyclins (See, Hunter, T. and J. Pines, Cell 79:573-582 (1994); Grana, X. and E. P. Reddy, Oncogene 11:211-219 (1995)).

Transgenic experiments have been used to study the forced expression of cyclin D1 in specific cell types. Results have varied dependent upon the cell type. Expression of an MMTV-LTR-cyclin D1 transgene led to constitutive mammary hyperplasia (Wang, T. C. et al, Nature (Lond.) 369:669-671 (1994)). In contrast, no lymphocyte hyperplasia was observed in mice carrying an Eμ-cyclin D1 transgene, although mice carrying both Eμ-cyclin D1 and Eμ-myc transgenes exhibited accelerated lymphoma formation as compared with mice with the Eμ-myc transgene alone (See, Bodrug, S. E. et al., Eur. Mol. Biol. Organ. J. 13:2124-2130 (1994); and Lovec, H. et al., Eur. Mol. Biol. Organ. J. 13:3487-3495 (1994)). Mice carrying a MHC-cyclin D1 transgene exhibit multinucleation and sustained DNA synthesis in adult cardiomyocytes as measured by tritiated thymidine incorporation assays (Soonpaa, M. H. et al., J. Clin. Invest. 99:2644-2654 (1997)).

In view of this background, there remains a need for additional strategies for enhancing the proliferative potential of cells such as cardiomyocytes, and for use of proliferatively-enhanced cells. The present invention addresses these needs.

### SUMMARY OF THE INVENTION

A feature of the present invention involves the discovery that increasing cyclin D2 activity in cardiomyocyte cells provides enhanced proliferative potential to the cells. Accordingly, one aspect of the invention concerns a method for enhancing the proliferative potential of a cardiomyocyte cell, comprising increasing the level of cyclin D2 activity in the cardiomyocyte cell. In one form, this may involve introducing nucleic acid into the cardiomyocyte cells, wherein the nucleic acid has a sequence of nucleotides encoding cyclin D2. Such introduction can be carried out with the cell in vitro or in vivo, and where in vitro the modified cell can in one utility thereafter be grafted into a mammal, including a human.

Another aspect of the invention provides a cardiomyocyte cell having introduced nucleic acid encoding cyclin D2, the cardiomyocyte exhibiting increased proliferative potential. The cell may for example have introduced nucleic acid having a coding sequence corresponding to nucleotides 4 to 870 of SEQ. I.D. NO. 1 or of SEQ. I.D. NO. 3 in the Sequence Listing, or having a coding sequence sufficiently similar thereto to encode a protein having cyclin D2 activity. The nucleotide sequence may be operably linked to a promoter, including for example a constitutive promoter, an inducible promoter or a cardiomyocyte-specific promoter.

In another aspect, the invention provides nucleic acid constructs including a sequence of nucleotides encoding cyclin D2 operably linked to a promoter such as an inducible promoter or a cardiomyocyte-specific promoter. The cyclin D2 coding sequence may correspond to nucleotides 4 to 870 of SEQ. I.D. NO. 1 or of SEQ. I.D. NO. 3, or may be a sequence of nucleotides sufficiently similar thereto to encode a protein having cyclin D2 activity.

The present invention also provides a method for increasing the proliferative potential of myocardial cells in a mammal. This method involves increasing the level of cyclin D2 activity in cardiomyocytes in myocardial tissue of the mammal, so as to result in an increased proliferative potential. For example, cardiomyocytes within myocardial tissue can be genetically transduced with an expression vector incorporating nucleic acid encoding cyclin D2 operably linked to a promoter such as a constitutive, inducible or cardiomyocyte-specific promoter.

The invention herein also concerns a method for grafting cardiomyocytes in a mammal. The method includes grafting cardiomyocytes or cardiomyogenic cells into a mammal, wherein the cardiomyocytes exhibit an increased level of cyclin D2 activity and have increased proliferative potential. The grafted cells may have introduced nucleic acid encoding cyclin D2 operably linked to a promoter such as a constitutive, inducible or cardiomyocyte-specific promoter.

Also provided by the invention are methods for inducing an increase in the proliferative potential of cardiomyocytes in myocardial tissue of a mammal. The methods include providing cardiomyocytes in myocardial tissue of the mammal, wherein the cardiomyocytes are responsive to a pharmacologic agent to increase the proliferative potential of the cardiomyocytes. The agent is administered to the mammal so as to achieve an increase in the proliferative potential of the cardiomyocytes. The inducible cardiomyocytes may for instance be provided as grafted inducible cells within the myocardial tissue, or may result from an in vivo genetic transduction of existing cells in the myocardial tissue.

In another embodiment, the present invention provides a modified D-type cyclin, wherein the cyclin has been modified to remove one or more (and potentially all) phosphorylation sites present in its native form.

The present invention provides cardiomyocyte cells having enhanced proliferative capacity, and methods and materials for making and using such cells. Additional embodiments and features of the invention will be apparent from the descriptions herein.

### DESCRIPTION OF THE FIGURES

FIG. 1 is a schematic diagram showing a map of the MHC-CYCD2 transgene prepared as described in Example 1.

FIG. 2 presents the results of a Western blot analysis of cyclin D2 expression in the hearts of control mice (−) and transgenic mice (lines designated 10, 9, 5 and 3) carrying the transgene shown in FIG. 1, prepared as described in Example 2.

FIGS. 3A and 3B are photomicrographs presenting the results of a pulse chase experiments demonstrating cardiomyocyte DNA synthesis and kariokinesis, respectively, in vivo in MHC-CYCD2 mice in response to a pharmacologic stimulus, as further described in Example 3.

FIG. 4 provides a bar graph showing increased cell numbers in the left and right atria of transgenic MHC-CYCD2 mice as compared to nontransgenics, generated as described in Example 7.

FIG. 5 provides a bar graph illustrating that culture of cardiomyocytes from the left atria of MHC-CYCD2 transgenic mice in the presence of isoproterenol leads to an increase in the number of cardiomyocyte nuclei in the culture, as described in Example 8.

FIGS. 6A and 6B provide digital images of transgenic MHC-CYCD2 cardiomyocytes undergoing cytokinesis, obtained as described in Example 9.

FIGS. 7A and 7B provide photomicrographs illustrating DNA synthesis of cardiomyocytes in transgenic MHC-CYCD2 mice in a cautery injury model emulating infarction, obtained as described in Example 10.

FIG. 8 provides a schematic diagram of a STK-rMHC-Switch-CycD2 virus, as described in Example 11 below.

FIG. 9 provides a schematic diagram of a STK-rMHC-CycD2-nLAC virus, as described in Example 12 below.

### DESCRIPTION OF THE PREFERRED EMBODIMENTS

For the purpose of promoting an understanding of the principles of the invention, reference will now be made to certain preferred embodiments thereof and specific language will be used to describe the same. It will nevertheless be understood that no limitation of the scope of the invention is thereby intended, such alterations, further modifications and applications of the principles of the invention as described herein being contemplated as would normally occur to one skilled in the art to which the invention relates.

As described herein, it has been discovered that increasing the level of cyclin D2 activity can be used to provide to a cell, e.g. a generally non-proliferative cell, such as a mammalian cardiomyocyte, an enhanced proliferative potential. The invention makes available, inter alia, novel cells with enhanced proliferative potential, novel methods involving the use of such cells in vivo or in vitro, novel genetic constructs and methods useful for modifying cells to obtain cells of enhanced proliferative potential, novel cellular grafting methods, and novel animal models having such cells.

Cyclin D2 proteins of mammalian origin, including for example the mouse and human proteins, are known. U.S. Pat. No. 5,869,640, issued Feb. 9, 1999, discloses amino acid and nucleotide sequences for D-type cyclins, including cyclin D2 proteins, as well as characterizing data, and is hereby incorporated herein by reference in its entirety. Cyclin D2 is structurally related to but distinct from the other known D-type cyclins, cyclin Dl and cyclin D3. These D-type cyclins bind to and activate CDK4 and CDK6. This protein complex then phosphorylates members of the retinoblastoma family, thereby releasing E2F family members (which are normally bound to and thereby inhibited by hypophosphorylated RB family members). Released E2F initiates cell cycle progression by promoting the transcription of a variety of gene products needed for DNA synthesis.

Basic structural properties of known native D-type cyclins are presented in Table 1 below:

TABLE 1

| | Molecular | | |
|---|---|---|---|
| Cyclin | Weight (daltons) | # Amino Acids | Phosphorylation Sites (#) |
| D1 | 33,426 | 295 | cAMP Kinase (1) |
| | | | Ca Kinase (1) |
| | | | CKII Kinase (1) |
| | | | GSK3 Kinase (6) |
| D2 | 32,849 | 289 | GSK 3 Kinase (3) |
| D3 | 32,408 | 292 | cAMP Kinase (3) |
| | | | Ca Kinase (3) |
| | | | GSK3 Kinase (4) |

Many prior reports have suggested that these three cyclins are functionally redundant. However, the discoveries herein reveal that significant functional differences exist between cyclin D2 and cyclins D1 and D3. Illustratively, Table 2 below provides a comparison of ventricular and left atrial DNA synthesis measured in hearts of transgenic cyclin D2 mice to that in corresponding transgenic cyclin D1 and D3 mice. In each case, testing was performed generally as described in Example 3 below (HW/BW=heart weight/body weight; Iso=isoproterenol treated). The cautery injury (C.I.) data (emulative of infarct) were obtained using procedures generally as described in Example 10 below.

TABLE 2

| | HW/BW | Ventricular DNA Synth. (%) | | | Left Atrial DNA Synth. (%) | |
|---|---|---|---|---|---|---|
| Mice | (% sibs) | Baseline | Iso(7 days) | C.I. | Baseline | Iso(7 days) |
| Cyclin D1 | 136.9 ± 10.58 | 0.12 | 0.00 | 0.015 | 0.00 | 0.00 |
| (n) | | (20021) | (25060) | (139000) | (˜25000) | (˜16000) |
| Cyclin D2 | 120.2 ± 4.98 | 0.20 | 0.12 | 0.53 | 0.31 | 7.28 |
| (n) | | (35029) | (32007) | (3203) | (18311) | (22706) |
| Cyclin D3 | 130.9 ± 7.52 | 0.22 | 0.00 | 0.01 | 0.00 | 0.00 |
| (n) | | (22005) | (25425) | (˜25000) | (˜25000) | (˜16000) |
| Control | 100 | 0.0005 | 0.00 | 0.0083 | 0.00 | 0.00 |
| (n) | | (180000) | (˜200000) | (36000) | (18000) | (18000) |

As can be seen, DNA synthesis did not cease in response to treatment with isoproterenol in the transgenic cyclin D2 mice, whereas it did in the transgenic cyclin D1 and D3 mice. In addition, DNA synthesis in the transgenic cyclin D2 mice increased in response to cautery injury (see ventricular data above) and treatment with isoproterenol (left atrial data). Accordingly, cyclin D2 exhibits functional characteristics distinct from those of cyclins D1 and D3.

A comparison of the amino acid sequence of cyclin D2 to those of D1 and D3 reveals several domains of substantial difference. For example, D2 differs significantly from D1 in domains occurring at about amino acid residues 200-240 and 260-280. D2 differs significantly from D3 in domains occurring at about amino acid residues 210-225 and 250-280. Thus, cyclin D2 differs from both cyclins D1 and D3 in a region spanning about nucleotides 200-280. Functionally, these cyclins differ in their propensity for phosphorylation sites, as illustrated in Table 1. As expected, many of these sites reside within the domains of non-homology identified above.

SEQ. I.D. NO. 1 shows the nucleotide sequence and deduced amino acid sequence for mouse cyclin D2 as utilized in the Examples herein (see also Genbank Accession No. 83749 for the mouse cyclin D2 sequence). SEQ. I.D. NO. 3 shows the nucleotide sequence and deduced amino acid sequence for human cyclin D2. In this regard, the term “nucleotide sequence,” as used herein, is intended to refer to a natural or synthetic sequential array of nucleotides and/or nucleosides, and derivatives thereof. The term amino acid sequence is intended to refer to a natural or synthetic sequential array of amino acids and/or derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

It will be understood that the present invention also encompasses the use of nucleotide sequences and amino acid sequences which differ from the specific cyclin D2 sequences disclosed herein, but which have substantial identity thereto and thereby exhibit characteristic cyclin D2 activity as identified herein. Such sequences will be considered to provide cyclin D2 nucleic acid and cyclin D2 proteins for use in the various aspects of the present invention. For example, nucleic acid sequences encoding variant amino acid sequences are within the scope of the invention. Modifications to a sequence, such as deletions, insertions, or substitutions in the sequence, which produce “silent” changes that do not substantially affect the functional properties of the resulting polypeptide molecule are expressly contemplated by the present invention. For example, it is understood that alterations in a nucleotide sequence which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

Also, phosphomimetic mutations such as substitution of serine for aspartic acid in a serine-specific protein kinase consensus sequence can be expected to produce a product mimiking a constitutively phosphorylated Cyclin D2 product.

Nucleotide changes which result in alteration of the N-terminal and C-terminal portions of the encoded polypeptide molecule would also not generally be expected to alter the activity of the polypeptide. In some cases, it may in fact be desirable to make mutations in the sequence in order to study the effect of alteration on the biological activity of the polypeptide. Each of the proposed modifications is well within the routine skill in the art.

In one manner of defining the invention, nucleic acid (e.g. DNA) may be used that has a coding sequence that differs from that set forth in SEQ. I.D. NO. 1 (nucleotides 4-870) or from that set forth in SEQ. I.D. NO. 3 (nucleotides 4-870), wherein the nucleic acid, or at least the coding portion thereof, will bind to nucleic acid having nucleotides 4-870 of SEQ. I.D. NO. 1 or SEQ. I.D. NO. 3 under stringent conditions, and which nucleic acid encodes a polypeptide having cyclin D2 activity. “Stringent conditions” are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C.

In one preferred aspect, the encoded polypeptide will retain phosphorylation site characteristics consistent with those of the native cyclin D2 polypeptide, having fewer phosphorylation sites than native cyclin D1 (9 sites) and D3 (10 sites), and/or lacking cAMP kinase, Ca kinase, and/or CKII kinase phosphorylation sites, and/or containing only GSK3 kinase phosphorylation site(s). Furthermore, cyclin D2 may be modified in accordance with the present invention using site directed mutagenesis to reduce the number of, or eliminate completely, its phosphorylation sites. Additionally, cyclins Dl and D3 may be modified to reduce the number of, or eliminate completely, their phosphorylation sites using site directed mutagenesis, to arrive at D-type cyclins that more closely emulate cyclin D2 in regard to phosphorylation capacity. Such modifications can be achieved, for example, by eliminating phosphorylatable amino acids such as serine and threonine, and replacing them with non-phosphorylatable amino acids, preferably non-charged, non-polar amino acids such as alanine which do not detrimentally impact the conformation of the protein. Cyclins D1 and D3 may also be modified to replace one or more other regions of non-homology with cyclin D2 with corresponding D2 regions, to provide composite D-type cyclins exhibiting functional characteristics similar to those demonstrated by cyclin D2 herein. These and/or other potential modifications to native D-type cyclins to provide modified D-type cyclins having characterizing activities consistent with those demonstrated by cyclin D2 herein (e.g. maintained DNA synthesis in response to insult and/or inducibility) are contemplated as forming a part of the present invention.

In another manner of defining the invention, nucleic acid may be used that encodes a polypeptide that has an amino acid sequence which has at least about 70% identity, more preferably at least about 80% identity, most preferably a least about 90% identity, with the amino acid sequence set forth in SEQ. I.D. NO. 2, or SEQ. I.D. No. 4 or with at least one significant length (i.e. at least 40 amino acid residues) segment thereof, and which polypeptide possesses cyclin D2 activity. The polypeptide may, for example, have an amino acid sequence which has at least about 70%, 80%, or 90% identity with amino acid residues 200-280 of SEQ. I.D. NO. 2 or SEQ. I.D. No. 4, which represent a region in which cyclin D2 differs from cyclins Dl and D3. Percent identity, as used herein, is intended to mean percent identity as determined by comparing sequence information using the advanced BLAST computer program, version 2.0.8, available from the National Institutes of Health, USA. The BLAST program is based on the alignment method of Karlin and Altschul, Proc. Natl. Acad. Sci. USA 87:2264-68 (1990) and as discussed in Altschul, et al., J. Mol. Biol. 215:403-10 (1990); Karlin and Altschul, Proc. Natl. Acad. Sci. USA 90:5873-7 (1993); and Altschul et al. (1997) Nucleic Acids Res. 25:3389-3402. Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Preferred default parameters for the BLAST program, blastp, include: (1) description of 500; (2) Expect value of 10; (3) Karlin-Altschul parameter λ=0.270; (4) Karlin-Altschul parameter K=0.0470; (5) gap penalties: Existence 11, Extension 1; (6) H value=4.94e^{-324;} (6) scores for matched and mismatched amino acids found in the BLOSUM62 matrix as described in Henikoff, S. and Henikoff, J. G., Proc. Natl. Acad. Sci. USA 89:10915-10919 (1992); Pearson, W. R., Prot. Sci. 4:1145-1160 (1995); and Henikoff, S. and Henikoff, J. G., Proteins 17:49-61 (1993). The program also uses an SEG filter to mask-off segments of the query sequence as determined by the SEG program of Wootton and Federhen Computers and Chemistry 17:149-163, (1993).

In another form, nucleic acid may be used that includes a coding sequence that has at least about 70% identity with the coding portion of the nucleotide sequence set forth in SEQ. I.D. NO. 1 or SEQ. I.D. NO. 3 (nucleotides 4 to 870), or with at least one significant length (i.e. at least 100 nucleotides) segment thereof, and which nucleic acid encodes a polypeptide possessing characteristic cyclin D2 activity as identified herein. The nucleic acid may, for example, have a coding sequence which has at least about 70% at least about 80%, or at least about 90%, identity with nucleotides 601 to 843 (coding for amino acids 200-280) of SEQ. I.D. NO. 1 or SEQ. I.D. NO. 3.

The nucleotide sequence may be operably linked to a promoter sequence as known in the art to provide recombinant nucleic acid useful in a variety of applications including, for example, in the provision of vehicles such as vectors for functionally introducing the nucleic acid in to mammalian or other eukaryotic cells. As defined herein, a nucleotide sequence is “operably linked” to another nucleotide sequence (e.g. a regulatory element such as a promoter) when it is placed into a functional relationship with the other nucleotide sequence. For example, if a nucleotide sequence is operably linked to a promoter sequence, this generally means that the nucleotide sequence is contiguous with the promoter and the promoter exhibits the capacity to promote transcription of the gene. A wide variety of promoters are known in the art, including cell-specific promoters, inducible promoters and constitutive promoters. The promoters may be selected so that the desired product produced from the nucleotide sequence template is produced constitutively in the target cells. Alternately, promoters such as inducible promoters may be selected that require activation by activating elements known in the art, so that production of the desired product may be regulated as desired. Still further, promoters may be chosen that promote transcription of the gene in one or more selected cell types, e.g. the so-called cell-specific promoters.

In a preferred aspect of the invention, the cyclin D2 nucleotide sequence is operably linked to a cardiomyocyte cell-specific promoter, for example, providing for constitutive expression of the nucleotide sequence in cardiomyocytes. Illustrative candidates for such promoters include the α-myosin heavy chain (α-MHC) promoter, the β-myosin heavy chain (β-MHC) promoter, the myosin light chain-2V (MLC-2V) promoter, the atrial natriuretic factor (ANF) promoter, and the like. Such constructs enable the expression of the cyclin D2 nucleic acid selectively in cardiomyocyte cells.

Another aspect of the invention provides recombinant nucleic acid that includes a cyclin D2 nucleotide sequence operably linked to an inducible promoter, such that cyclin D2 expression and enhancement of the proliferative capacity of cells incorporating the nucleic acid can be upregulated in response to an inducing agent. Illustrative candidate inducible promoter systems include, for example, the metallothionein (MT) promoter system, wherein the MT promoter is induced by heavy metals such as copper sulfate; the tetracycline regulatable system, which is a binary system wherein expression is dependent upon the presence or absence of tetracycline; a glucocorticoid responsive promoter, which uses a synthetic sequence derived from the glucocorticoid response element and is inducible in vivo by administering dexamethasome (cells having the appropriate receptor); a muristerone-responsive promoter, which uses the gonadotropin-releasing hormone promoter and is inducible with muristerone (cells having the appropriate receptor); and TNF responsive promoters. Additional inducible promoters which may be used, and which are more preferred, include the ecdysone promoter system, which is inducible using an insect hormone (ecdysone) and provides complete ligand-dependent expression in mammals; the β-GAL system, which is a binary system utilizing an E. coli lac operon operator and the I gene product in trans, and a gratuitous inducer (IPTG) is used to regulate expression; and, the RU486 inducible system, which uses the CYP3A5 promoter and is inducible by RU486, a well defined pharmaceutical agent. These and other similar inducible promoter systems are known, and their use in the present invention is within the purview of those skilled in the area.

The present invention also concerns vectors which incorporate a cyclin D2 nucleotide sequence and which are useful in the genetic transduction of myocardial cells in vitro or in vivo. A variety of vector systems are suitable for these purposes. These include, for example, viral vectors such as adenovirus vectors as disclosed for example in Franz et al., Cardiovasc. Res. 35(3):560-566 (1997); Inesi et al., Am. J. Physiol. 274 (3 Pt. 1):C645-653 (1998); Kohout et al., Circ. Res. 78(6):971-977 (1996); Leor et al., J. Mol. Cell Cardiol. 28(10):2057-2067 (1996); March et al., Clin. Cardiol. 22(1 Suppl. 1):123-29 (1999); and Rothman et al., Gene Ther. 3(10):919-926 (1996). Adeno-Associated Virus (AAV) vectors are also suitable, and are illustratively disclosed in Kaptlitt et al., Ann. Thora. Surg. 62(6):1669-1676 (1996); and Svensson et al., Circulation 99(2):201-205 (1999). Additional viral vectors which may be used include retroviral vectors (see e.g. Prentice et al., J. Mol. Cell Cardiol. 28(l):133-140 (1996); and Petropoulos et al., J. Virol. 66(6):3391-3397 (1992)), and Lenti (HIV-1) viral vectors as disclosed in Rebolledo et al., Circ. Res. 83(7):738-742 (1998). A preferred class of expression vectors will incorporate the cyclin D2 nucleic acid operably linked to a cardiomyocyte-specific promoter, such as one of those identified above. Still further, AAV vectors are highly compatible for use in transfection of myocardial cells and tissue, and are preferred from among those identified above.

In accordance with the invention, cardiomyocytes can also be genetically transduced with cyclin D2 nucleic acid in vitro or in vivo using liposome-based transduction systems. A variety of liposomal transduction systems are known, and have been reported to successfully deliver recombinant expression vectors to cardiomyocytes. Illustrative teachings may be found for example in R. W. Zajdel, et al., Developmental Dynamics. 213(4):412-20 (1998); Y. Sawa, et al., Gene Therapy.5(11):1472-80 (1998); Y. Kawahira, et al., Circulation 98(19 Suppl):II262-7; discussion II267-8 (1998); G. Yamada, et al., Cellular & Molecular Biology 43(8):1165-9 (1997); M. Aoki, et al., Journal of Molecular & Cellular Cardiology 29(3):949-59 (1997); Y. Sawa, et al., Journal of Thoracic & Cardiovascular Surgery 113(3):512-8; discussion 518-9 (1997); and I. Aleksic, et al., Thoracic & Cardiovascular Surgeon 44(2):81-5 (1996). Thus, liposomal recombinant expression vectors including cyclin D2 DNA can also be utilized to tranduce cardiomyocytes in vitro and in vivo for the purposes described herein.

Nucleic acid constructs can be used for example to introduce nucleotide sequences encoding a cyclin D2 protein into cardiomyocyte cells in vivo or in vitro, to achieve a level of intracellular cyclin D2 activity that is increased relative to the native level of the cardiomyocyte cells. Such increased activity can provide an enhanced proliferative capacity to the cells. An enhanced proliferative capacity can be evidenced, for example, by an increase in the level of DNA synthesis and nuclear number (kariokinesis), and/or the exhibition of increased levels of cytokinesis or cell division and consequent increases in cell number. DNA synthesis can be monitored in conventional fashion, for example by tritiated thymidine incorporation analysis. Cytokinesis can also be conventionally detected, e.g. by standard cell counting techniques in vitro or in vivo or generally by the observation of increased cell mass or density correlated to increased cell numbers. Alternatively or in addition, purified (e.g. purified recombinant) cyclin D2 protein may be introduced into cells to increase cyclin D2 activity (e.g. by fusogenic liposomes or other macromolecular delivery systems), or the cells can be treated with pharmacologic agents which increase cyclin D2 activity, to provide increased proliferative potential to the cells.

The present invention makes available methods which can be applied in vitro or in vivo for research, therapeutic, screening or other purposes. Methods for the in vitro culture of cardiomyocytes expressing introduced cyclin D2 DNA can be used, for example, in the study and understanding of the cell cycle, in screening for chemical or physical agents which modulate cyclin D2 activity or other aspects of the cell cycle, or in the culture of cardiomyocyte cells for subsequent engraftment into a mammal, including humans.

Cardiomyocyte cells to be cultured in accordance with the invention can be derived from a variety of sources. For example, they may be harvested from a mammal for culture and subsequent engraftment into that mammal (autografts) or another mammal of the same species (allografts) or a different species (xenografts). The cardiomyocyte cells may also be derived from the differentiation of stem cells such as embryonic stem cells, or other similar pluripotent cells such as somatic stem cells that differentiate to cardiomyocytes. General methodology for such derivations is disclosed in U.S. Pat. Nos. 5,602,301 and 5,733,727 to Field et al. In this regard, when so derived, the genetic modification to incorporate the cyclin D2 nucleic acid may take place at the stem cell level, for instance utilizing one or more vectors to introduce the cyclin D2 nucleic acid operably linked to a cardiomyocyte-specific promoter, and nucleic acid enabling the selection of cardiomyocytes from other cells differentiating from the stem cell and/or at a differentiated level e.g., including a selectable marker gene operably linked to a cardiomyocyte—specific promoter. Nucleic acid enabling selection of transformed from non-transformed stem cells may also be used in such strategies. Such selection of the stem and/or cardiomyocyte cells may be achieved, illustratively, utilizing a gene conferring resistance to an antibiotic (e.g. neomycin or hygromycin) or other chemical agent operably linked to an appropriate promoter or by using a reporter operably linked to an appropriate promoter allowing for selection of cells by fluroescense activated cells sorting (FACS), for example the known GFP reporter.

Using stem-cell derived cardiomyocytes, the genetic modification to incorporate the cyclin D2 and potentially other nucleic acid may also occur after differentiation of the stem cells. For example, a differentiated cell population enriched in cardiomyocytes, for instance containing 90% or more cardiomyocytes, may be transformed with a vector having cyclin D2 nucleic acid operably linked to a promoter (optionally cardiomyocyte specific), as described above. The same or a different vector may also be used to introduce other functional nucleic acid to the cells, for example providing a reporter gene and/or selectable marker, or providing for the expression of a growth factor and/or another cell cycle regulatory protein.

In one mode of carrying out the invention, left ventricular, right ventricular, left atrial, or right atrial cardiomyocytes, or a mixture of some or all of these, may be genetically modified in vitro to incorporate functional cyclin D2 nucleic acid using a suitable vector as disclosed above. Cells to be genetically transduced in such protocols may be obtained for instance from animals at different developmental stages, for example fetal, neonatal and adult stages. Suitable animal sources include mammals such as bovine, porcine, equine, ovine and murine animals. Human cells may be obtained from human donors or from a patient to be treated. The modified cardiomyocytes may thereafter be implanted into a mammal, for example into the left or right atrium or left or right ventricle, to establish a cellular graft in the mammal. Implantation of the cells may be achieved by any suitable means, including for instance by injection or catheterization. In addition to the cyclin D2 nucleic acid, the cells may also be modified in vitro to contain other functional nucleic acid sequences which can be expressed to provide other proteins, for example growth factors such as nerve growth factors, or angiogenic factors such as vascular endothelial growth factor-1 (VEGF-1), or one or more additional cell cycle regulatory proteins or other proteins which act as co-factors with cyclin D2 in increasing cellular proliferative potential.

Cardiomyocyte cells for culture, and potential implantation, may also be obtained from the heart of a transgenic animal (especially mammal) expressing introduced cyclin D2 nucleic acid. Using known techniques, transgenic animals which harbor introduced cyclin D2 nucleic acid in essentially all of their cells can be raised, and used either as a source for harvesting culturable cardiomyocyte cells or as animal models for research or screening purposes. For instance, transgenic bovine, porcine, equine, ovine or murine animals may be used as sources for the cardiomyocyte cells or as animal models for study.

The present invention also provides for the genetic modification of cardiomyocytes in vivo to introduce functional cyclin D2 nucleic acid. An expression vector containing cyclin D2 nucleic acid, for instance one as described above, may be delivered to myocardial tissue of a recipient mammal, to achieve transduction of cardiomyocytes in the tissue. In preferred modes, the cyclin D2 nucleic acid in such vector will be operably linked to a cardiomyocyte-specific promoter. The delivery of the vector can be suitably achieved, for instance, by injection, catheterization, or infusion into the blood stream. It will be understood that any mode of delivery which enables the establishment of transduced cardiomyocytes within the myocardial tissue of the recipient mammal is contemplated as being within the present invention. A single delivery of the vector may be used, or multiple deliveries nearly simultaneous or over time may be used, in order to establish a substantial population of transduced cells within the recipient. The transduced cells will then express the cyclin D2 protein, for instance under the control of a constitutive, inducible or cardiomyocyte-specific promoter, and thereby be reactivated to the cell cycle and exhibit an enhanced proliferative potential.

The implantation of cardiomyocytes or cardiomyogenic stem cells (e.g. genetically transduced stem cells as discussed herein) cultured in vitro or the delivery of the vector for in vivo genetic transduction may be directed or may home to a selected site or sites within the heart of the recipient. Such site or sites may be in the left or right atrium or left or right ventricle of the recipient, or any combination of these. Commonly, the implantation or delivery site or sites will occur in the left or right ventricle of the recipient. The site(s) may, for instance, be one(s) in which there is a need for additional viable cells, for example in a damaged or diseased area of the heart such as in cases of myocardial infarcts and cardiomyopathies. The site(s) may also be targets for the delivery of other proteins such as growth factors, e.g. nerve growth or angiogenic factors, as discussed above, via expression in the grafted or in vivo transduced cells.

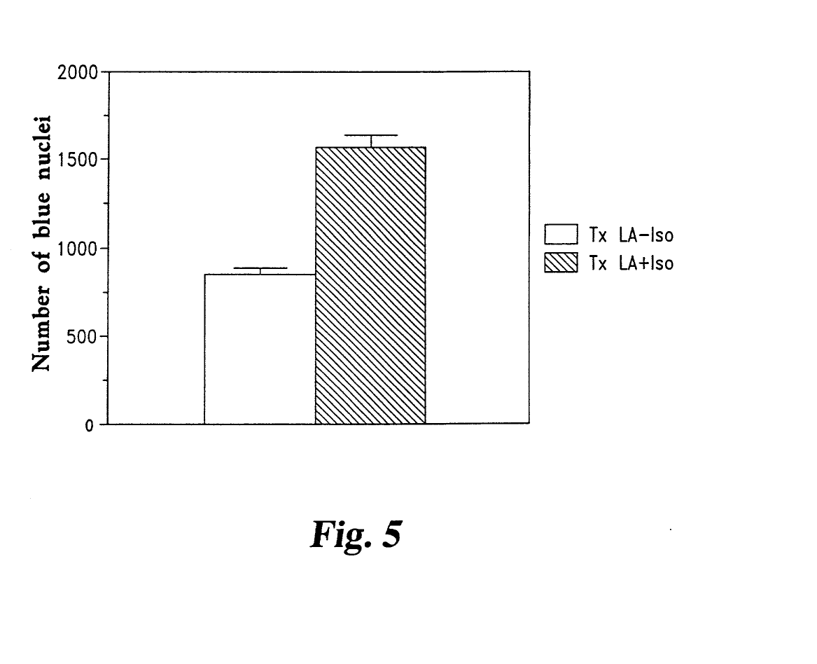

In another aspect of the invention, it has been discovered that cardiomyocytes having increased cyclin D2 activity can be provided which, in response to contact with a pharmacologic agent, exhibit a substantial increase in proliferative potential. For example, such increases in proliferative potential have been observed in the hearts of transgenic mice carrying cyclin D2 DNA linked to a cardiomyocyte specific promoter, as described in Examples 2 and 3 below. In this particular work, increases in proliferative potential in response to treatment with isoproterenol were observed in the left and right atria of the transgenic mice. In the right atriam of transgenic mice without in vivo isoproterenol treatment, the labeling index (thymidine incorporation analysis) was 0.09%. In corresponding mice with isoproterenol treatment, the right atrial labeling index was 0.29%. Dramatically, in the left atrium, the labeling index was 0.31% without in vivo isoproterenol treatment, and 7.28% with isoproterenol treatment. Still further, as discussed in Example 8 below and illustrated in FIG. 5, the culture of left atrial cardiomyocytes harvested from the transgenic cyclin D2 mice in the presence of isoproterenol provided a substantial increase in observed nuclei in culture. These surprising discoveries provide access to methods in which the proliferative potential of cardiomyocytes can be increased in vitro or in vivo utilizing enhanced cyclin D2 activity in combination with administration of or treatment with a suitable agent. In this regard, illustrative candidate agents for these purposes include pharmacologic agents, for example α-adrenergic and/or β-adrenergic receptor agonists, some of which are known to be hypertrophic agents, such as isoproterenol, epinephrine, norepinephrine, phenylephrine, and cyclic AMP inducing agents, such as forskolin, and other pharmacological agents which increase levels of endogenous proteins or other factors having similar functions. Given the teachings herein, these and other pharmacologic agents may be readily screened and identified for their capacity to increase the proliferative potential of cardiomyocyte cells having enhanced cyclin D2 activity.

In one utilization of this discovery, cellular engraftment techniques can capitalize upon the increased cardiomyocyte proliferative potential in response to the agent. For instance, the agent may be incorporated in the culture medium during culture of the cells for subsequent implantation in the heart, and/or the cells after implantation can be treated with the agent continuously or periodically to sustain the increased proliferative potential. In another utilization, cardiomyocytes in the heart of a mammal may be treated in vivo to enhance their cyclin D2 activity, and then the agent can be administered to the mammal to achieve an increase in proliferative potential.

Cellular engraftment and/or in vivo genetic modification in accordance with the invention can be used, for example, to deliver therapy to mammals, including humans. A variety of ex vivo cellular transplantation and implantation techniques and gene therapy techniques are thus contemplated as forming a part of the invention. These may be used to target an improvement of the contractile function of the heart of the patient, for example in the treatment of contractile losses due to infarcts or cardiomyopathies.

The present discoveries also provide access to methods for screening the activity of biologic, pharmacologic or other agents upon cardiomyocytes using cells of the invention. For example, access is provided to screening for co-factors or other conditions which, in combination with the enhanced cyclin D2 activity, lead to increased cardiomyocyte proliferative potential as a baseline or in response to treatment with an agent. For example, the differential response of the heart chambers of the transgenic cyclin D2 mice described herein may be due to the presence of a co-factor in the left atrium that is not present or has a reduced presence in the right atrium or ventricles, and/or to the presence of an inhibitory protein in the right atrium or ventricles that is not present or has a reduced presence in the left atrium. The transgenic cyclin D2 mice described herein enable the use of automated techniques to discover the presence or absence, or relative levels, of such co-factors or inhibitory proteins in the various chambers of the heart. The identity of the cofactor(s) can be established, for example, based on its differential pattern of expression in responding versus non-responding cardiac samples using established techniques. For example, gene chip technology, differential display, and subtractive hybridization approaches, among others, can be exploited to identify those gene products which are differentially expressed in the responsive versus non-responsive cardiac tissue. The use of cardiomyocyte-enriched samples, as well as analogous samples from non-transgenic tissue, would permit screening against those non-specific factors which are also differentially expressed (i.e. those expressed in non-cardiomyocytes, and those which are generically induced in proliferating cells, respectively). Ventricular and/or right atrial cardiomyocytes can then be modified to enhance their ability to respond to agents as do the left atrial cardiomyocytes. For example, right atrial or right or left ventricular cardiomyocytes can be modified (e.g. transformed) in vitro or in vivo to increase expression of one or more proteins which are co-factors for cyclin D2 in responding to the agent, or can be so modified to decrease expression of inhibitory factors. In this manner, additional agent-responsive, proliferatively-enhanced cardiomyocytes are provided.

### EXAMPLES

For the purpose of promoting a further understanding of the principles and features of the present invention, the following specific Examples are provided. It will be understood that these Examples are intended to be illustrative, and not limiting, of the invention.

### EXAMPLE 1

### Preparation of a MHC-CYCD2 Fusion Gene

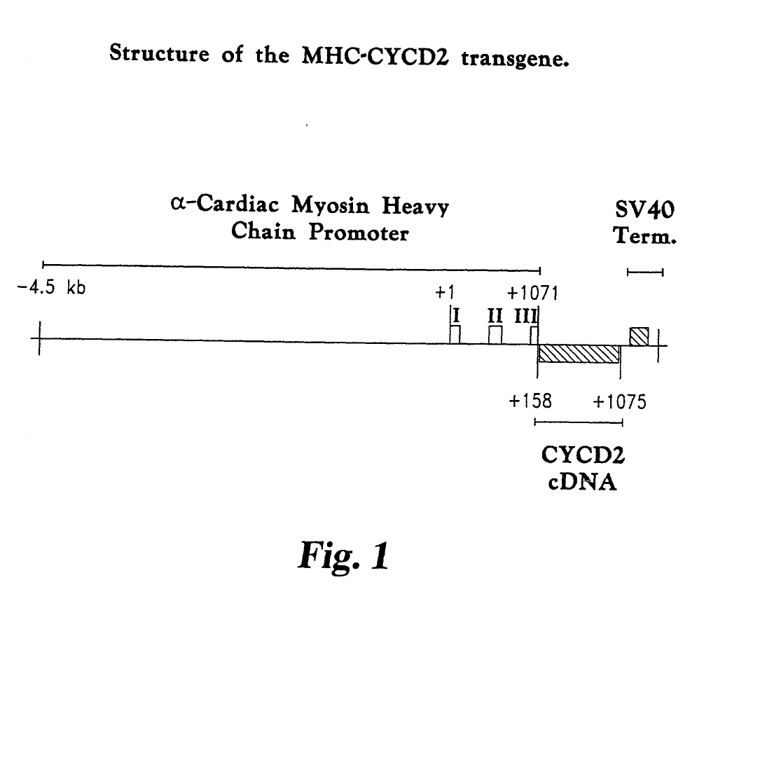

A MHC-CYCD2 transgene was constructed using the transcriptional regulatory sequences of the mouse α-cardiac myosin heavy chain (MHC) gene and a cDNA encoding mouse cyclin D2 (CYCD2) protein. The MHC promoter (SEQ. I.D. NO. 5) consisted of 4.5 kb of 5′ flanking sequence and 1 kb of the gene encompassing exons 1-3 up to but not including the initiation codon (Gulick, J. et al., J. Biol. Chem. 266:9180-9185 (1991)). The CYCD2 cDNA encompassed nucleotide residues #268-1143; Genbank Accession #M83749) (SEQ. I.D. No. 1), and was generated by reverse transcriptase-polymerase chain reaction (RT-PCR) amplification of mouse heart RNA as described (Kim, K. K. et al., J. Biol. Chem. 269:22607-22613 (1994)). The integrity of the CYCD2 cDNA was confirmed by sequence analysis. The sequence of the sense primer was 5′ GCT ATG GAG CTG CTG TGC TGC GAG GTG GAC 3′ (SEQ. I.D. No. 7). The sequence of the antisense primer was 5′ TCC TCA CAG GTC AAC ATC CCG CAC GTC TGT 3′ (SEQ. I.D. No. 8). The SV40 early region transcription terminator/polyadenylation site (nucleotide residues 2586-2452) was inserted downstream from the CYCD2 cDNA insert. The resulting transgene, designated MHC-CYCD2, was digested with Nru I and transgene insert was purified by agarose gel elecrophoresis and eluted with Geneclean glass beads. A map of the transgene is provided in FIG. 1.

### EXAMPLE 2

### Generation of MHC-CYCD2 Transgenic Mice

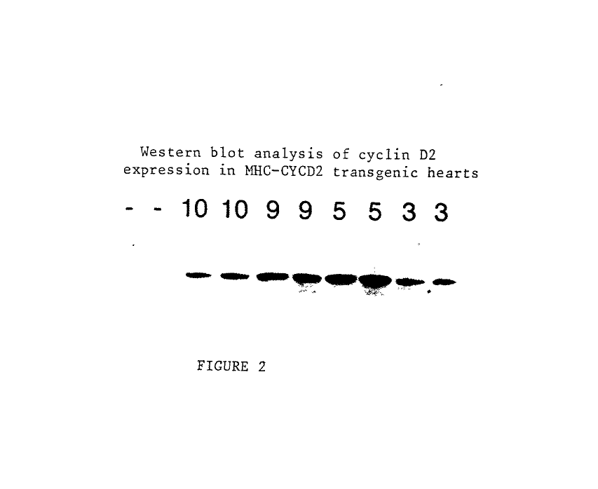

The MHC-CYCD2 insert prepared in Example 1 was purified and injected into one cell embryos following standard procedures (Hogan, B., Manipulating the Mouse Embryo, Plainview, N.Y. Cold Spring Harbor Laboratory Press, p. 497 (1994). The resulting 34 mice were screened for the presence of the transgene, and 11 were identified as being transgenic. No obvious morbidity was apparent in the founder mice. Eight mice randomly selected and placed in breeding cages ultimately gave rise to 4 transgenic lineages. Transgene expression was initially established by Western blot analysis (FIG. 2). Heart homogenate was prepared from non-transgenic adult heart (−) as well as adult mice from MHC-CYCD2 lines designated 10, 9, 5 and 3. Samples from two individual mice from each lineage were analyzed. Hearts were homogenized in NP40 buffer (150 mM NaCl, 5 mM EDTA, 50 mM Tris-HCl pH 8.0, 1 μg/ml aprotinin, 1 μg/ml pepstatin, 1 μg/ml leupeptin, 50 μg/ml TLCK, 50 μg/ml PMSF, 100 μg/ml TPCK, 1% vol/vol Nonidet P-40). The homogenate was cleared by centrifugation at 40,000× g for 10 min, and the protein content of the supernatant was quantitated using a commercial assay (Bio-Rad, Richmond Calif.). Samples (60 μg/lane) were separated by size on 10% polyacrylamide gels under denaturing conditions, and electro-blotted to nitrocellulose (Hoefer Scientific, San Francisco Calif.) membranes. The filters were stained with 0.1% naphthol blue-black in 45% methanol, 10% acetic acid to assess the efficiency of transfer. For Western analysis, nonspecific binding was blocked by incubation in block buffer (5% nonfat dry milk, 3% BSA, 0.1% Tween, 1×PBS) for 2 hr at room temperature. The antibody used in this study was a rat monoclonal antibody against cyclin D2 (Oncogene Science) at a working concentration of 2.5 μg/ml). Western blot analyses revealed that high levels of cyclin D2 protein were present in the hearts of the adult transgenic mice. Other Western blot analyses failed to detect elevated levels of cyclin D2 in all other tissues examined, consistent with the known myocardial specificity of the MHC promoter. EXAMPLE 3

### Demonstration of Increased Cardiomyocyte DNA Synthesis

A thymidine incorporation assay was used to determine if cardiomyocyte DNA synthesis persisted in adult transgenic MCH-CYCD2 animals. This testing also employed a second transgenic mouse line, designated MHC-nLAC. The MCH-nLAC mice express a nuclear localized β-galactosidase (βGAL) reporter gene exclusively in the cardiomyocytes (Soonpaa, M. H. et al., Science 264:98-101 (1994); Soonpaa, M. H. and L. J. Field, Am. J. Physiol. 272:H220-226 (1997)). Accurate cardiomyocyte tritiated thymidine labeling indices can be readily obtained with the MHC-nLAC animals simply by screening for co-localization of βGAL activity and silver grains in autoradiographs of 5-bromo-4-chloro-3-indolyl-B-D-galactoside (X-GAL) stained heart sections. To monitor the effect of cyclin D2 overexpression on cardiomyocyte DNA synthesis, MHC-CYCD2 mice were crossed with MHC-nLAC mice and animals carrying either the MHC-nLAC transgene alone or both transgenes were identified and sequestered. When the mice reached 11 weeks of age, they received a single injection of tritiated thymidine and were sacrificed four hours later. The hearts were removed, sectioned, stained with X-GAL and processed for autoradiography. Ventricular cardiomyocyte labeling indices of 0.24% were observed for the double transgenic mice, whereas no DNA synthesis was observed in the MHC-nLAC control group (about 30,000 nuclei were scored for each group, n=5 mice). DNA synthesis was also observed in the atria of the double transgenic mice, with the results presented in Table 3 below. In light of the sustained cardiomyocyte DNA synthesis observed in the adult MHC-CYCD2 mouse hearts, a series of experiments was initiated to ascertain how the transgenic myocardium would respond to cardiac hypertrophy. Osmotic mini-pumps (Model 2001, Alzet, Palo Alto, Calif., flow rate of 1 μl per hour) filled with saline or 0.028 g/ml isoproterenol in saline were implanted through a small longitudinal incision between the scapulae. 8 control mice (MHC-nLAC) and 8 cyclin D2 mice (MHC-nLAC/MHC-CYCD2 double transgenics) were used. In the cyclin expressing mice, continuous administration of isoproterenol for 7 days resulted in a 47.6% increase in heart weight/body weight. In control mice, isoproterenol treatment resulted in a 28% increase in heart weight/body weight as compared to saline treated animals.

Prior to sacrifice, the experimental and control mice received a bolus injection of tritiated thymidine to permit assessment of cardiomyocyte DNA synthesis. After a 4 hour chase, the animals were sacrificed, and the hearts were harvested, cryoprotected, sectioned, stained with X-GAL and subjected to autoradiography. Once again, cardiomyocyte DNA synthesis was measured by scoring the presence of silver grains over βGAL positive nuclei. A huge increase in the left atria cardiomyocyte labeling index was observed in the MHC-CYCD2 mice following isoproterenol treatment (0.31% for the non-isoproterenol-treated group versus 7.28% for the isoproterenol-treated group). DNA synthesis in the right atrium of MHC-CYCD2 mice was moderately increased, and in the ventricle of these mice was moderately decreased. Isoproterenol treatment had no effect on cardiomyocyte DNA synthesis in the non-cyclin expressing control group.

TABLE 3

| Mice | Right Atrium | Left Atrium | Ventricle |
|---|---|---|---|
| Control | | | |
| Uninjured | 0% | 0% | 0% |
| Isoproterenol | 0% | 0% | 0% |
| MHC-CYCD2 | | | |
| Uninjured | 0.09% | 0.31% | 0.24% |
| Isoproterenol | 0.29% | 7.28% | 0.11% |

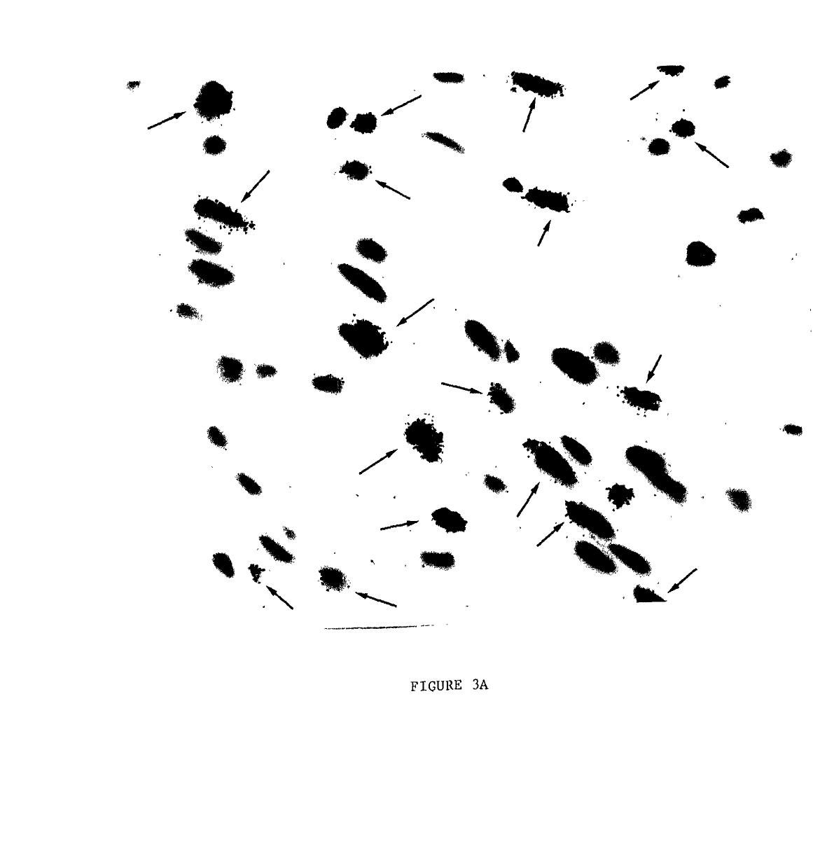

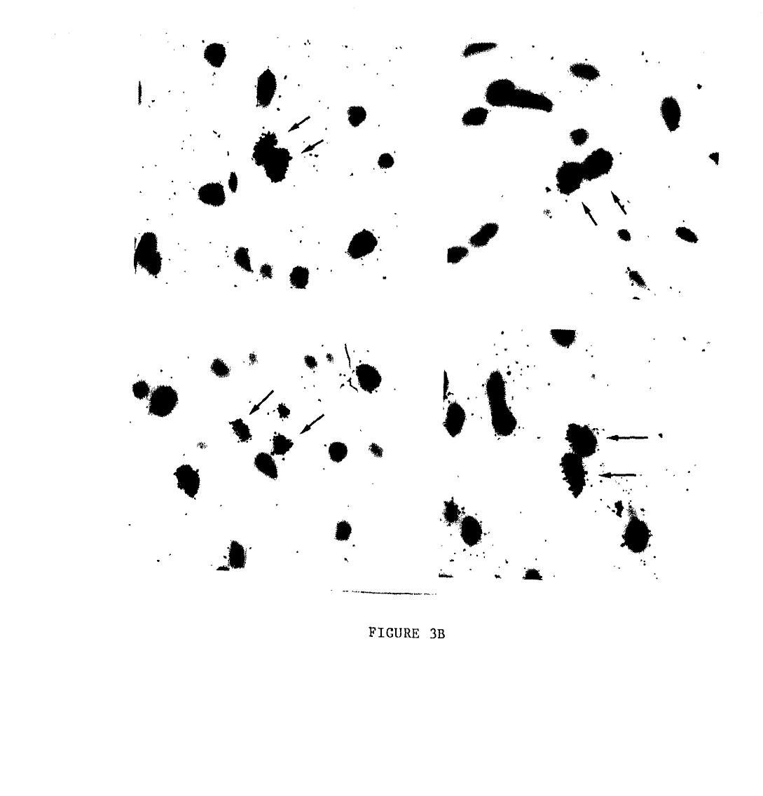

The above data demonstrate that transgenic animals expressing cyclin D2 have sustained atrial and ventricular cardiomyocyte DNA synthesis, and that the rate of atrial cardiomyocyte DNA synthesis is dramatically increased in response to the administration of isoproterenol. Pulse chase experiments were employed to determine the fate of the cardiomyocytes synthesizing DNA. Once again, MHC-CYCD2 mice were crossed with MHC-nLAC mice. The MHC-nLAC mice express a nuclear localized β-GAL reporter exclusively in cardiomyocytes. Mice from this cross carrying either the MHC-nLAC transgene alone or both the MHC-nLAC and MHC-CYCD2 transgenes were identified and sequestered. At 11 weeks of age, myocardial hypertrophy was induced by isoproterenol infusion with Alzet minipumps (minipump model 2001, Alzet, Palo Alto Calif.; flow rate of 1 μl/hr, 0.028 g/ml isoproterenol). After 7 days of isoproterenol infusion, the control (MHC-nLAC) and experimental (MHC-nLAC/MHC-CYCD2 double transgenic) mice received a single injection of 3H-thymidine (200 uCi I.P. at 28 Ci/mM, Amersham, Arlington Heights, Ill.), and were sacrificed either 4 hours (pulse, FIG. 3A) or 72 hours (chase, FIG. 3B) later. The hearts were removed, cryoprotected in 30% sucrose, embedded and sectioned at 10 μm using standard histologic techniques. The sections were post-fixed in formaldehyde:glutaraldehyde (1:1) and overlaid with 1 mg/ml X-GAL, 5 mM potassium ferricyanide, 5 mM potassium ferrocyanide and 2 mM magnesium chloride in PBS. The sections were counter-stained with DAPI, and washed three times in PBS. After drying, stained slides were coated with photographic emulsion (Ilford L.4, Polysciences, Warrington Pa.) diluted 1:1 with water, drained, and placed in a light-tight box for four days at 4° C. Slides were then developed in Kodak D-19 (Rochester N.Y.) for four minutes, washed in water, and fixed in 30% sodium thiosulfate for at least four minutes. Slides were further processed by washing in H₂O and by dehydration through graded ethanols and xylene, followed by application of a coverslip. Cardiomyocyte DNA synthesis was scored by the co-localization of βGAL activity (blue staining) and silver grains. The high DNA synthesis labeling index seen following the three day chase period indicated that the cardiomyocytes which undergo DNA synthesis were viable (in contrast to pronounced apoptosis observed with E1A and E2F gene transfer into cardiomyocytes, see Kirshenbaum et al., J. Biol. Chem. 270:7791-7794 (1995); Kirshenbaum et al., Dev. Biol. 179:402-411 (1996)). Cardiomyocyte DNA synthesis following isoproterenol-induced hypertrophy in MHC-CYCD2/MHC-nLAC transgenic mice is evident from the presence of silver grains over blue nuclei (arrows, FIG. 3A). In FIG. 3B, the appearance of silver grains over paired blue nuclei is indicative of DNA synthesis followed by nuclear division (or kariokinesis, see paired arrows).

### EXAMPLE 4

### Analysis of Levels of Various Proteins in MHC-CYCD2 Transgenic Mice

Western blots were used to analyze protein expression levels in adult MHC-CYCD2 mice and their non-transgenic litter mates. Hearts were homogenized in NP40 buffer (150 mM NaCl, 5 mM EDTA, 50 mM Tris-HCl pH 8.0, 1 μg/ml aprotinin, 1 μg/ml pepstatin, 1 μg/ml leupeptin, 50 μg/ml TLCK, 50 μg/ml PMSF, 100 μg/ml TPCK, 1% vol/vol Nonidet P-40). The homogenate was cleared by centrifugation at 40,000× g for 10 min, and the protein content of the supernatant was quantitated using a commercial assay (Bio-Rad, Richmond Calif.). Samples were separated by size on 10% polyacrylamide gels under denaturing conditions as described, and electro-blotted to nitrocellulose (Hoefer Scientific, San Francisco Calif.) membranes. The filters were stained with 0.1% naphthol blue-black in 45% methanol, 10% acetic acid to assess the efficiency of transfer. For Western analysis, nonspecific binding was blocked by incubation in block buffer (5% nonfat dry milk, 3% BSA, 0.1% Tween, 1×PBS) for 2 hr at room temperature. Commercial antibodies were used for each protein analyzed. Conditions (i.e. dilution, length of reaction, secondary antibody, etc) were according to the manufacturer's recommendations. The results are presented in Table 4 below, in which higher numbers of the symbol “+” indicate higher levels of protein, and the symbol “−” indicates none detected.

TABLE 4

| Marker | Nontransgenic | Transgenic |
|---|---|---|
| Cyclin D2 | + | + + + + + + + + + + |
| Cyclin D1 | + | + |
| Cyclin D3 | + | + |
| PCNA | + | + + + |
| CDC2 | − | − |
| CDK2 | + | + |
| CDK4 | + | + + + + |
| CDK6 | + | + |
| Dmp 1 | + | + |
| pRb | − | + + + |
| p107 | + | + + |
| p130 | + | + + |

These results demonstrate that upregulation of cyclin D2 in the transgenic mice was sufficient to elicit increased expression in a number of gene products required for cell cycle progression.

### EXAMPLE 5

### Analysis of Cell Cycle Protein Level Changes in Response to Isoproterenol

Myocardial hypertrophy was induced by isoproterenol infusion with Alzet minipumps in adult MHC-CYCD2 mice and their non-transgenic siblings (minipump model 2001, Alzet, Palo Alto Calif.; flow rate of 1 μl/hr, 0.028 g/ml isoproterenol). Hearts were harvested after 7 days of isoproterenol infusion and processed for Western blot analysis using procedures as described in Example 4. The results are presented in Table 5 below. Again, higher numbers of the symbol “+” indicate higher levels of protein, and the symbol “−” indicates none detected.

TABLE 5

| | NonTransgenic | NonTransgenic | Transgenic | Transgenic |
|---|---|---|---|---|
| Marker | (−Iso) | (+Iso) | (−Iso) | (+Iso) |
| Cyclin D2 | + | + | + + + + + | + + + + + |
| | | | + + + + + | + + + + + |
| PCNA | + | + + | + + | + + + + |
| CDC2 | − | + + | − | + + + + |

### EXAMPLE 6

### Culture of CYCD2 Versus Control Cardiomyocytes

Hearts from MHC-CYCD2 transgenic mice or their non-transgenic siblings were harvested at the age indicated, and the left atria were dissected and digested in PBS (37° C., 60 min) containing 0.17% collagenase (Type I, Worthington Biochemical, Freehold N.J.). Cells were then triturated with a Pasteur pipette and plated at a density of 1×10⁵ cells per chamber slide in DMEM medium containing 10% FBS supplemented with 1 μm isoproterenol. Plating was scored by the presence or absence of contractile cells 72 hours later. The results are presented in Table 6 below, in which “+” indicates a successful culture and indicates an unsuccessful culture.

TABLE 6

| Postnatal Stage | Nontransgenic | Transgenic |
|---|---|---|
| Day 1 | + | + |
| Day 8 | − | + |
| Day 14 | − | + |
| Day 21 | − | + |

These results demonstrate that increased cyclin D2 activity can be used to achieve dramatic improvement in the capacity to culture cardiomyocyte cells.

### EXAMPLE 7

### Demonstration of Increased Cell Numbers in Left and Right Atria

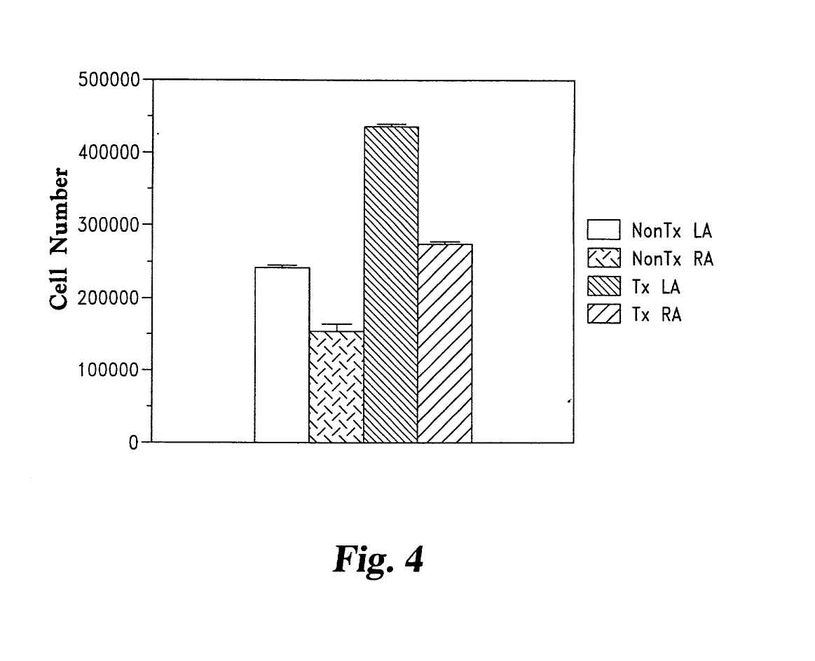

Left and right atria from neonatal day 14 MHC-CYCD2 transgenic mice and their non-transgenic siblings were harvested and digested in PBS (37° C., 60 min) containing 0.17% collagenase (Type I, Worthington Biochemical, Freehold N.J.). Cells were then triturated with a Pasteur pipette and counted directly with a hemocytometer. The results are graphically represented in FIG. 4, which shows increased cell numbers in the left and right atria of MHC-CYCD2 mice as compared to nontransgenics.

### EXAMPLE 8

### Demonstration of Increased Cell Nuclei (Kariokinesis) in Cultures with Isoproterenol

Left atria from neonatal day 14 MHC-CYCD2 transgenic mice were harvested and digested in PBS (37° C., 60 min) containing 0.17% collagenase (Type I, Worthington Biochemical, Freehold N.J.). Cells were then triturated with a Pasteur pipette and plated at a density of 1×10⁵ cells per chamber slide. Cells were cultured in DMEM supplemented with 10% FBS. In some cases, the media also contained isoproterenol (1 μm). After 72 hrs., the slides were fixed in gluteraldehyde-formaldehyde (1:1) and overlaid with 1 mg/ml X-GAL, 5 mM potassium ferricyanide, 5 mM potassium ferrocyanide and 2 mM magnesium chloride in PBS. The number of blue nuclei were counted directly on a microscope. FIG. 5 provides a bar graph of the results, showing that culture of cardiomyocytes from the left atria of MHC-CYCD2 transgenic mice in the presence of isoproterenol leads to a substantial increase in the number of cardiomyocyte nuclei in the culture.

### EXAMPLE 9

### Demonstration of Cell Proliferation (Cytokinesis) in CYCD2 Cardiomyocytes

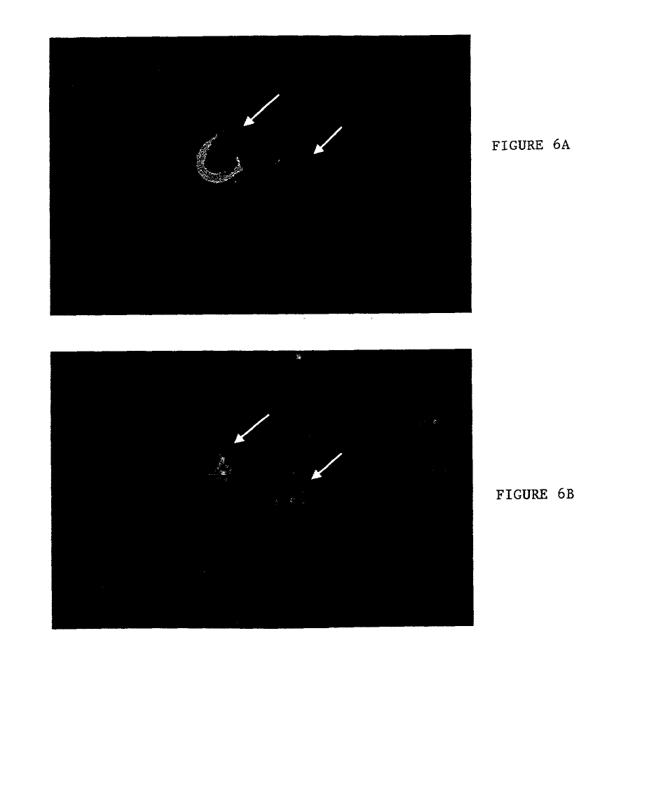

Left atria from neonatal day 14 MHC-CYCD2 transgenic mice were harvested, digested, triturated, plated and cultured in DMEM supplemented with 10% FBS and isoproterenol (1 μm), as described in Example 8. After 72 hrs., the slides were fixed in acetone and processed for myosin heavy chain immune reactivity using monoclonal antibody MF-20. Signal was developed using a FITC-conjugated anti-mouse IgG secondary antibody. Nuclei were counter stained with Hoechst 3334. FIG. 6A shows a cardiomyocyte undergoing cytokinesis (FITC signal, green cube); FIG. 6B shows the same field though for Hoechst staining (blue cube).

### EXAMPLE 10

### Demonstration of Cell Cycle Activation in Cautery Injury Model

In this Example it was demonstrated that the cell cycle of cardiomyocytes with increased cyclin D2 levels is activated in a cautery injury model which mimics myocardial infarction. 11 week old MHC-nLAC control or MHC-nLAC/MHC-CYCD2 double transgenic mice were anesthetized (2.5% Avertin, 0.015 ml/g body weight, I.P., Fluka Chemicals, Ronkomkoma N.Y.) and intubated (Small Animal Respirator, 70 cycles/second, tidal pressure 1.2 kpascals, Narco Biosystems, Houston Tex.). The heart was exposed via an incision at the third intercostal space, and the myocardium was cauterized midway between the apex and base of the heart using a Medi-Pak surgical cautery (General Medical Corporation Richmond Va.). After cauterization, the incision was closed, the pneumothorax evacuated, and the mice allowed to recover from anesthesia on a heating pad maintained at 37° C. The mortality rate for the procedure was <5%. All animal manipulations were performed in accordance with institutional guidelines. 7 days after injury, the experimental and control mice received a single injection of 3H-thymidine (200 μgCi I.P. at 28 Ci/mM, Amersham, Arlington Heights, Ill.), and were sacrificed 4 hours later. The hearts were removed, cryoprotected in 30% sucrose, embedded and sectioned at 10 μm using standard histologic techniques. To localize regions of myocardial damage, sections were stained with Hemotoxylin and Eosin (H and E) according to the manufacturer's specifications (Sigma). To localize cardiomyocyte nuclei, sections were post-fixed and overlaid with 1 mg/ml X-GAL, 5 mM potassium ferricyanide, 5 mM potassium ferrocyanide and 2 mM magnesium chloride in PBS. The sections were counter-stained with DAPI, and after drying autoradiographic emulsion (Ilford L.4, Polysciences, Warrington Pa.) diluted 1:1 with water, drained, and placed in a light-tight box for 4 days at 4° C. Slides were then developed in Kodak D-19 (Rochester N.Y.) for four minutes, washed in water, and fixed in 30% sodium thiosulfate for at least four minutes. Slides were further processed by washing in H₂0 and by dehydration through graded ethanols and xylene, followed by application of a coverslip.

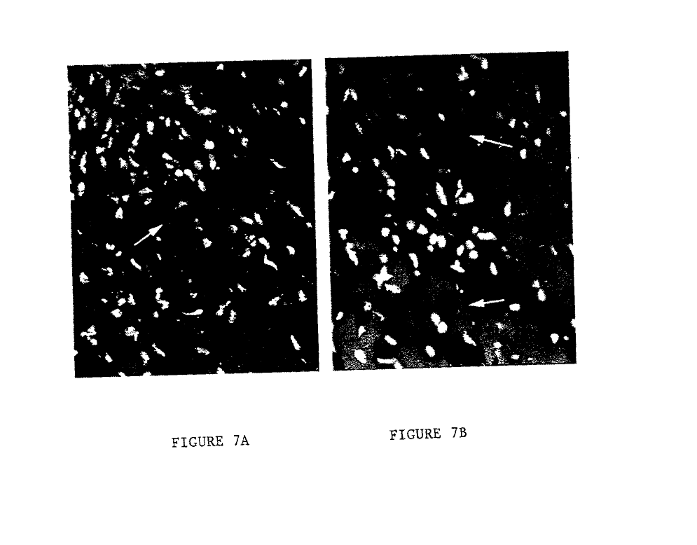

Cardiomyocyte DNA synthesis in MHC-CYCD2 transgneic mice was also monitored following cautery injury, which mimmics myocardial infarction. The left ventricular free wall was injured by cauterization. Gross examination of the hearts 7 days post-injury revealed the presence of a necrotic zone at the site of cauterization. In addition, pronounced blanching of the myocardium was evident in the region distal to and apically located from the cauterization site. The appearance and location of the blanching was consistent with ischemic myocardial damage resulting from disruption of the underlying vasculature at the site of cauterization. The extent of myocardial damage was readily detected in histologic sections; as much as 50% of the left ventricular free wall was affected. To monitor DNA synthesis, injured MHC-nLAC transgenic animals (controls) and injured MHC-nLAC/MHC-CYCD2 transgenic animals received a single injection of tritiated thymidine. The hearts were then harvested, sectioned, stained with X-GAL and processed for autoradiography. FIG. 7A shows a single synthetic ventricular cardiomyocyte nucleus (arrow) in the peri-necrotic zone of an MHC-nLAC/MHC-CYCD2 transgenic mouse located apically from the cauterization site. FIG. 7B shows the peri-necrotic zone from a different MHC-nLAC/MHC-CYCD2 transgenic animal; the arrows point to two cardiomyocyte nuclei undergoing DNA synthesis. 0.53% of the cardiomyocytes in the peri-necrotic zone were synthesizing DNA in the MHC-nLAC/MHC-CYCD2 transgenic animals (3,202 cells were screened). In contrast, 0% of the cardiomyocytes in the peri-necrotic zone were synthesizing DNA in the MHC-nLAC control animals (3,400 cells were screened). Thus, an increase in cardiomyocyte DNA synthesis is observed in the cyclin D2 expressing hearts in response to injury. Moreover, the overall rate of cardiomyocyte DNA synthesis in the MHC-CYCD2 hearts is in vast excess to that in the injured control hearts (which was undetectable in the assay performed).

### EXAMPLE 11

### STK-rMHC-Switch-CycD2 Virus

This Example describes a virus designed to provide inducible expression of cyclin D2 in adult cardiac tissue or cardiomyocytes useful for engraftment. The known STK virus is utilized. STK is a 3rd generation Adenovirus which has been modified so as not to encode any Adenoviral proteins. This design limits any host immune response against cells transduced with the virus in vivo.

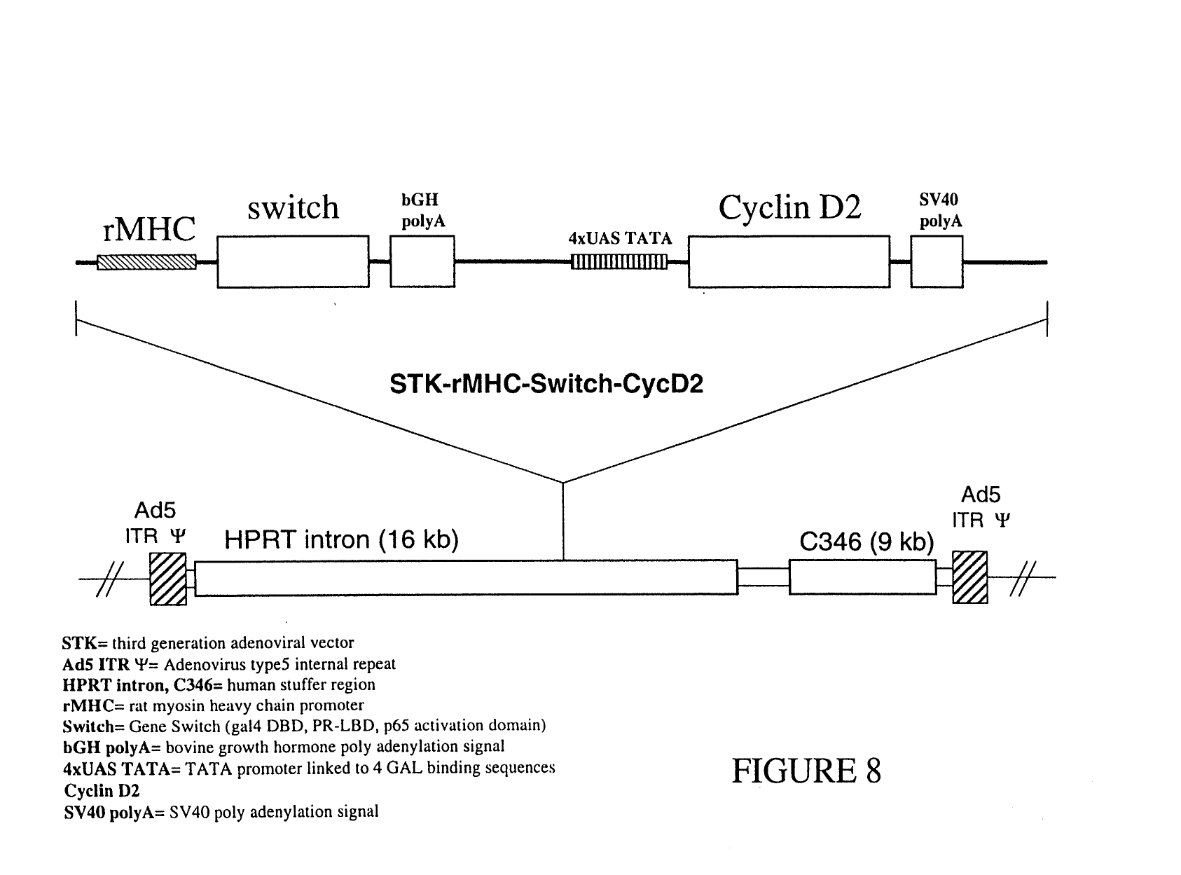

With reference to FIG. 8, the virus contains two transcriptional units. The first transcriptional unit utilizes the rat alpha-cardiac myosin heavy chain (rMHC) promoter to target cardiac specific expression of the known “Gene-Switch” transcription factor. The polyadenylation and transcription termination sequences from the bovine growth hormone (bGH) gene is inserted down-stream of the Gene-Switch sequence. Cardiomyocytes transfected with this virus will express the Gene-Switch protein. In contrast non-cardiomyocytes transfected with this virus will not express the “Gene-Switch” protein, as the rMHC promoter is not active in non-cardiomyocytes. The Gene-Switch transcription factor is only active in the presence of an appropriate ligand (as for example Ru486).

The second transcriptional unit in the virus utilizes a 4×UAS TATA promoter to target expression of cyclin D2 (CycD2). The polyadenylation and transcription termination sequences from the SV40 early region is inserted down-stream. from the CycD2 sequence. Transcription from the 4×UAS TATA promoter is dependent upon the presence of active Gene-Switch protein.

Thus constructed, the system is used and functions as follows. Heart tissue and/or cardiomyocytes to be used for engraftment are virally transduced with the STK-rMHC-Switch-CycD2 virus. Transfected cardiomyocytes express the Gene-Switch protein, which is inactive in the absence of ligand. Non-cardiomyocytes do not express the Gene-Switch protein. To activate the system, ligand is administered. This results in the activation of Gene-Switch transcription factor in cardiomyocytes. The activated Gene-Switch transcription factor initiates transcription at the 4×UAS TATA promoter. This in turn results in the synthesis of CycD2 mRNA, and ultimately CycD2 protein. Thus, the system provides for regulated synthesis of Cyclin D2. It will be used to direct gene expression (and consequently cell cycle activation) in adult cardiomyocytes.

### EXAMPLE 12

### Text for the STK-rMHC-CycD2-nLAC virus:

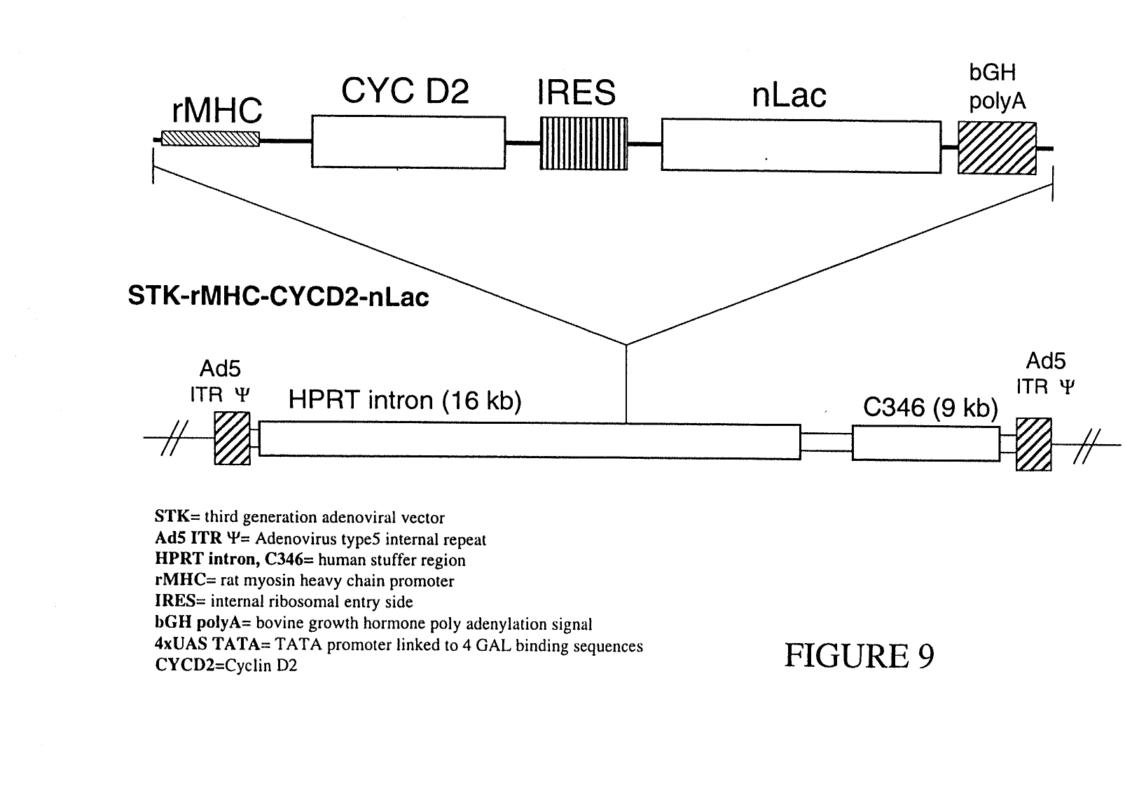

This Example describes the design of a virus useful to provide constitutive expression of cyclin D2 in adult cardiac tissue or other cardiomyocytes. The STK virus is utilized, as in Example 11 above. With reference now to FIG. 9, a single bi-cistronic transcriptional unit is utilized. The rat alpha-cardiac myosin heavy chain (rMHC) promoter (see American Journal of Physiology, Vol;. 262: H1867-H1876 (1992)) is used to target cardiac specific expression of Cyclin D2 (CycD2An internal ribosomal entry site is located downstream of the CycD2 sequences. This is followed by sequences encoding a marker gene (nLAC, a nuclear localized beta-galactosidase). Thus cardiomyocytes transfected with this virus will express a bi-cistronic transcript which encodes both the CycD2 and marker gene sequences. In contrast non-cardiomyocytes transfected with this virus will not express the bi-cistronic transcript, as the rMHC promoter is not active in non-cardiomyocytes. Thus, the system provides for constitutive synthesis of Cyclin D2 in adult cardiomyocytes. The presence of the marker gene will permit discrimination between infected and non-infected cardiomyocytes.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

All publications cited herein are indicative of the level of skill in the art and are hereby incorporated by reference as if each had been individually incorporated by reference and fully set forth.

What is claimed is:

1. A method for increasing the proliferative potential of a cardiomyocyte cell, comprising increasing the level of cyclin D2 activity in the cardiomyocyte cell so as to increase the proliferative potential of the cardiomyocyte cell.

2. The method of claim 1, comprising:
providing cardiomyocyte cells in a culture medium;
introducing nucleic acid into the cardiomyocyte cells, said nucleic acid having a sequence of nucleotides encoding a cyclin D2 protein; and
culturing the cardiomyocyte cells under conditions suitable for expression of said cyclin D2 protein.

3. The method of claim 1, wherein said introduced nucleic acid has a nucleotide sequence corresponding to nucleotides 4 to 870 of SEQ. I.D. NO. 1 or SEQ. I.D. NO. 3, or a nucleotide sequence having substantial identity thereto.

4. The method of claim 3, wherein said introduced nucleic acid has a nucleotide sequence corresponding to nucleotides 4 to 870 of SEQ. I.D. NO. 1 or SEQ. I.D. NO. 3, or said introduced nucleic acid hybridizes to nucleic acid having nucleotides 4 to 870 of SEQ. I.D. NO. 1 or SEQ. I.D. NO. 3 under stringent conditions, and encodes a protein having cyclin D2 activity.

5. The method of claim 4, wherein said nucleotide sequence is operably linked to a promoter.

6. The method of claim 4, wherein said promoter is a constitutive promoter.

7. The method of claim 4, wherein said promoter is an inducible promoter.

8. The method of claim 4, wherein said promoter is a cardiomyocyte specific promoter.

9. The method of claim 1, wherein said cardiomyocyte cell is a mammalian cardiomyocyte cell.

10. A method for culturing cardiomyocyte cells, comprising:
providing cardiomyocyte cells in a culture medium, said cardiomyocyte cells having an increased intracellular level of cyclin D2; and
culturing the cardiomyocyte cells in said culture medium.

11. The method of claim 10, wherein said cardiomyocyte cells have introduced nucleic acid encoding a cyclin D2 protein operably linked to a constitutive promoter.

12. The method of claim 10, wherein said cardiomyocyte cells have introduced nucleic acid encoding a cyclin D2 protein operably linked to an inducible promoter.

13. The method of claim 10, wherein said cardiomyocyte cells have introduced nucleic acid encoding a cyclin D2 protein operably linked to a cardiomyocyte specific promoter.

14. The method of claim 10, wherein said introduced nucleic acid is DNA having a nucleotide sequence corresponding to nucleotides 4 to 870 of SEQ. I.D. NO. 1 or SEQ. I.D. NO. 3, or a nucleotide sequence having substantial identity thereto.

15. The method of claim 10, wherein said introduced nucleic acid encodes a protein having the amino acid sequence of SEQ. I.D. NO. 2 or SEQ. I.D. NO. 4, or a polypeptide having an amino acid sequence at least 70% identical to the amino acid sequence of SEQ. I.D. NO. 2 or SEQ. I.D. NO. 4, and which exhibits cyclin D2 activity.

16. The method of claim 10, wherein said culturing includes culturing in the presence of a pharmacologic agent that induces an increase in the proliferative potential of the cells.

17. The method of claim 10, wherein said cardiomyocytes are mammalian cardiomyocytes.

18. The method of claim 10, wherein said cardiomyocytes are human cardiomyocytes.

19. The method of claim 10, wherein said cardiomyocytes are murine cardiomyocytes.

20. A cardiomyocyte cell having introduced nucleic acid encoding a cyclin D2 protein.

21. The cell of claim 20, wherein said introduced nucleic acid has a nucleotide sequence corresponding to nucleotides 4 to 870 of SEQ. I.D. NO. 1 or SEQ. I.D. NO. 3, or a nucleotide sequence having substantial identity thereto.

22. The cell of claim 21, wherein said introduced nucleic acid encodes a polypeptide having the amino acid sequence of SEQ. I.D. NO. 2 or SEQ. I.D. NO. 4, or a polypeptide having an amino acid sequence at least 70% identical to the amino acid sequence of SEQ. I.D. NO. 2 or SEQ. I.D. NO. 4, and which exhibits cyclin D2 activity.

23. The cell of claim 20, wherein said nucleotide sequence is operably linked to a promoter.

24. The cell of claim 23, wherein said promoter is a constitutive promoter.

25. The cell of claim 23, wherein said promoter is an inducible promoter.

26. The cell of claim 23, wherein said promoter is a cardiomyocyte specific promoter.

27. The cell of claim 20, wherein said cardiomyocyte cell is a mammalian cardiomyocyte cell.

28. The cell of claim 20, wherein said cardiomyocyte cell is a human cardiomyocyte cell.

29. A nucleic acid construct having a sequence of nucleotides encoding a cyclin D2 protein, said sequence of nucleotides operably linked to an inducible promoter.

30. The construct of claim 29, wherein said sequence of nucleotides corresponds to nucleotides 4 to 870 of SEQ. I.D. NO. 1 or SEQ. I.D. NO. 3, or is a sequence of nucleotides having substantial identity thereto.

31. The construct of claim 29, wherein said sequence of nucleotides corresponds to nucleotides 4 to 870 of SEQ. I.D. NO. 1 or SEQ. I.D. NO. 3, or is a sequence of nucleotides which hybridizes to nucleotides 4 to 870 of SEQ. I.D. NO. 1 or SEQ. I.D. NO. 3 under stringent conditions and which encodes a polypeptide having cyclin D2 activity.

32. A nucleic acid construct having a sequence of nucleotides encoding a cyclin D2 protein operably linked to a cardiomyocyte-specific promoter.

33. The construct of claim 32, wherein said sequence of nucleotides corresponds to nucleotides 4 to 870 of SEQ. I.D. NO. 1 or SEQ. I.D. NO. 3, or is a sequence of nucleotides having substantial identity thereto.

34. The construct of claim 32, wherein said sequence of nucleotides corresponds to nucleotides 4 to 870 of SEQ. I.D. NO. 1 or SEQ. I.D. NO. 3, or is a sequence of nucleotides which hybridizes to nucleotides 4 to 870 of SEQ. I.D. NO. 1 or SEQ. I.D. NO. 3 under stringent conditions and which encodes a protein having cyclin D2 activity.

35. A method for increase the proliferative potential of myocardial cells in a mammal, comprising:
increasing the level of cyclin D2 in cardiomyocytes in myocardial tissue of the mammal so as to increase the proliferative potential of the cardiomyocytes.

36. The method of claim 35, which comprises transfecting said cardiomyocytes with an expression vector having nucleic acid encoding a cyclin D2 protein operably linked to a promoter.

37. The method of claim 36, wherein said promoter is a constitutive promoter.

38. The method of claim 36, wherein said promoter is an inducible promoter.

39. The method of claim 36, wherein said promoter is a cardiomyocyte specific promoter.

40. The method of claim 35, also comprising administering to the mammal an agent that increases activation of the cell cycle in the transfected cardiomyocytes.

41. The method of claim 40, wherein the pharmacologic agent is an adrenergic receptor agonist.

42. The method of claim 41, wherein the receptor agonist is a β-adrenergic receptor agonist.

43. A method for providing proliferative cardiomyocytes in a mammal, comprising:
providing cardiomyocytes in a mammal, said cardiomyocytes responsive to an agent to increase the proliferative capacity of said cardiomyocytes; and
administering said agent to the mammal so as to increase the proliferative capacity of the cardiomyocytes.

44. The method of claim 43, wherein said cardiomyocytes contain introduced DNA encoding a cyclin D2 protein.

45. The method of claim 44, wherein said introduced DNA is operatively linked to an inducible promoter, and said agent causes induction of said inducible promoter.

46. A transgenic, non-human mammal having cardiomyocytes expressing introduced DNA encoding a cyclin D2 protein, the cardiomyocytes thereby exhibiting an activated cell cycle.

47. A modified D-type cyclin protein having removed therefrom one or more phosphorylation sites present in its native form, the modified D-type cyclin exhibiting the capacity to provide to mammalian cardiomyocytes an increased proliferative potential and sustained DNA synthesis when subjected to injurious stimuli.

48. A nucleic acid molecule encoding a modified D-type cyclin of claim 47.


SEQUENCE LISTING (8 sequences)

SEQ ID NO: 1 - DNA, 876 bp, Mus musculus
  CDS (4)..(870)
gctatggagctgctgtgctgcgaggtggacccggtccgcagggccgtgccggaccgcaacctgctggaagaccgcgttctgcagaacctgttgaccatcgaggagcgctacctcccgcagtgttcctatttcaagtgcgtgcagaaggacatccaaccgtacatgcgcaggatggtggccacctggatgctagaggtctgtgaggaacaaaagtgtgaagaagaggtctttcctctggccatgaattacctggaccgtttcttggctggagtcccgactcctaagacccatcttcagctcctgggtgcagtgtgcatgttcctagcttccaagctgaaagagaccatcccgctgactgcggaaaagctgtgcatttacaccgacaactctgtgaagccccaggagctgctggagtgggaactggtagtgttgggtaagctgaagtggaacctggccgcagtcacccctcacgacttcattgagcacatccttcgcaagctgccccagcaaaaggagaagctgtccctgatccgcaagcatgcgcagaccttcatcgctctgtgcgctaccgacttcaagtttgccatgtacccgccatcgatgattgcaactggaagcgtgggagcagccatctgtgggcttcagcaggatgatgaagtgaacacactcacgtgtgatgccctgactgagctgctggccaagatcacccacactgatgtggattgtctcaaagcctgccaggagcaaatcgaagctctgctgctgaacagcctgcagcagttccgtcaagagcagcataacgccggatccaagtctgtggaagatccggaccaagccaccacccctacagacgtgcgggatgttgacctgtgagga

SEQ ID NO: 2 - protein, 289 aa, Mus musculus
MELLCCEVDPVRRAVPDRNLLEDRVLQNLLTIEERYLPQCSYFKCVQKDIQPYMRRMVATWMLEVCEEQKCEEEVFPLAMNYLDRFLAGVPTPKTHLQLLGAVCMFLASKLKETIPLTAEKLCIYTDNSVKPQELLEWELVVLGKLKWNLAAVTPHDFIEHILRKLPQQKEKLSLIRKHAQTFIALCATDFKFAMYPPSMIATGSVGAAICGLQQDDEVNTLTCDALTELLAKITHTDVDCLKACQEQIEALLLNSLQQFRQEQHNAGSKSVEDPDQATTPTDVRDVDL

SEQ ID NO: 3 - DNA, 873 bp, Homo sapiens
  CDS (4)..(870)
gctatggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtga

SEQ ID NO: 4 - protein, 289 aa, Homo sapiens
MELLCHEVDPVRRAVRDRNLLRDDRVLQNLLTIEERYLPQCSYFKCVQKDIQPYMRRMVATWMLEVCEEQKCEEEVFPLAMNYLDRFLAGVPTPKSHLQLLGAVCMFLASKLKETSPLTAEKLCIYTDNSIKPQELLEWELVVLGKLKWNLAAVTPHDFIEHILRKLPQQREKLSLIRKHAQTFIALCATDFKFAMYPPSMIATGSVGAAICGLQQDEEVSSLTCDALTELLAKITNTDVDCLKACQEQIEAVLLNSLQQYRQDQRDGSKSEDELDQASTPTDVRDIDL

SEQ ID NO: 5 - DNA, 5443 bp, Mus musculus
ggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggcaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacatggaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgac

SEQ ID NO: 6 - DNA, 134 bp, Simian virus 40
gtggatgggcagcctatgattggaatgtcctctcaagtagaggaggttagggtttatgaggacacagaggagcttcctggggatccagacatgataagatacattgatgagtttggacaaaccacaactagaat

SEQ ID NO: 7 - DNA, 30 bp, Artificial Sequence
  Description of Artificial Sequence Primer
gctatggagctgctgtgctgcgaggtggac

SEQ ID NO: 8 - DNA, 30 bp, Artificial Sequence
  Description of Artificial Sequence Primer
tcctcacaggtcaacatcccgcacgtctgt